(19) United States
(12) Patent Application Publication
(10) Pub. No.: US 2010/0100024 A1
(43) Pub. Date: Apr. 22, 2010

(54) Anti-slip Mechanism, Garment, and Methods

(76) Inventor: Lawrence G. Reid, JR., Germanton, NC (US)

Correspondence Address: KILPATRICK STOCKTON LLP, 1001 WEST FOURTH STREET, WINSTON-SALEM, NC, 27101, US

(21) Appl. No.: 12/580,691

(22) Filed: Oct. 16, 2009

Related U.S. Application Data
(60) Provisional application No. 61/106,184, filed on Oct. 17, 2008.

(51) Int. Cl. A61L 15/00; A61F 13/00

(52) U.S. Cl. 602/63; 602/75

(57) ABSTRACT

An anti-slip garment can include a body and an anti-slip portion integrally formed with the body. The anti-slip portion can include a gripping ink composition applied on the inner surface of the anti-slip portion. In this way, when the garment is worn, the gripping ink frictionally engages a wearer's skin sufficiently to maintain the garment in a desired position on the wearer. In certain embodiments, the gripping ink can comprise a compound comprising silica in a ratio of about 1% to about 5% by weight. In such a garment, the risk of constriction due to elasticity in an area underlying the anti-slip portion can be prevented in the event swelling occurs or increases while the garment is being worn.

### CROSS-REFERENCE TO RELATED APPLICATION

This application claims benefit of U.S. Provisional Patent App. No. 61/106,184, filed Oct. 17, 2008, which application is incorporated by reference herein in its entirety.

### FIELD OF THE INVENTION

The present invention relates to an anti-slip mechanism, an anti-slip garment, methods of making an anti-slip garment, and/or methods of using an anti-slip mechanism and/or garment. Embodiments of the present invention may be useful for maintaining the position of a garment on a wearer.

### BACKGROUND

A lymphedema sleeve can cover an wearer's entire arm, forcing lymph fluid out of the arm. For some patients that have more severe edema and/or who are obese, a conventional lymphedema sleeve often may not remain positioned in a desired location on the larger upper arm. To overcome this problem, some conventional sleeves can incorporate a strap that connects to a bra strap or other article of clothing. Such straps can be costly to provide with a lymphedema sleeve, difficult to position, for example, due to recent surgery, and can be uncomfortable to the wearer.

Alternatively, some conventional lymphedema sleeves can include a band of material having silicone on the inside of the band and that is sewn onto the upper part of the sleeve. A disadvantage of a sewn-on band is that the stretch of the top of the sleeve where the band is located can be restricted due to the limited stretch of the band material and of the stitching to attach the band to the sleeve. As a result, the upper sewn band portion of the sleeve can create a constriction to flow of lymph fluid from more distal parts of the arm toward the proximal part of the arm and into the lymphatic system.

In some conventional medical device garments that encircle a wearer's body or limb, raw spandex has been added to the inside of the garment to provide gripping capability to the garment. One disadvantage of such a construction is that raw spandex can cause undesirable irritation when exposed to a wearer's skin, particularly over extended periods of time. Another disadvantage of a spandex-containing garment is that too much spandex can tighten around the wearer so as to restrict flow of fluids, including lymph fluid and/or blood. Conversely, not enough spandex in such a garment can result in the garment slipping down from its desired position on a wearer.

Thus, there is a need to provide an anti-slip garment that is adapted to be maintained in position on a wearer and that reduces the risk of constricting fluid flow. There is a need to provide such an anti-slip garment in which an anti-slip portion can be integrally formed with the garment. There is a need to provide such an anti-slip garment that allows the gripping capability of the anti-slip portion to be adjusted and that allows stretch properties of the anti-slip portion to be varied to accommodate various wearer circumferences. There is a need to provide such an anti-slip garment that is not irritating to a wearer.

### SUMMARY

Some embodiments of the present invention can include a garment anti-slip mechanism, an anti-slip garment, methods of making such an anti-slip garment, and/or methods of using such an anti-slip garment. Embodiments of the present invention may be useful for maintaining the position of a garment on a wearer.

In some embodiments, an anti-slip garment can comprise a body and an anti-slip portion integrally formed with the body. The anti-slip portion can include a gripping ink composition applied on the inner surface of the anti-slip portion. In this way, when the garment is worn, the gripping ink frictionally engages a wearer's skin sufficiently to maintain the garment in a desired position on the wearer. In certain embodiments, the gripping ink can comprise a compound comprising silica in a ratio of about 1% to about 5% by weight.

In some embodiments, the anti-slip portion can be located at or near an upper end of the anti-slip garment. In some embodiments, the anti-slip portion can include all or a portion of a welt. In some embodiments, the anti-slip garment can comprise a seamless garment. In certain embodiments, the garment body can include a first amount of elasticity and the anti-slip portion can include a second amount of elasticity that is equal to or less than the first amount of elasticity in the body. As a result, the risk of constriction due to elasticity in an area underlying the anti-slip portion can be prevented in the event swelling occurs or increases while the garment is being worn.

In some embodiments, the anti-slip garment can further comprise a lymphedema sleeve, a stocking, a compressive pressure garment, and/or other garment desired to be maintained in a particular position on a wearer.

Features of a garment anti-slip mechanism, an anti-slip garment, methods of making such an anti-slip garment, and/or methods of using such an anti-slip garment may be accomplished singularly, or in combination, in one or more of the embodiments of the present invention. As will be realized by those of skill in the art, many different embodiments of a garment anti-slip mechanism, an anti-slip garment, methods of making such an anti-slip garment, and/or methods of using such an anti-slip garment are possible. Additional uses, advantages, and features of aspects of the present invention are set forth in the illustrative embodiments discussed in the detailed description herein and will become more apparent to those skilled in the art upon examination of the following.

### BRIEF DESCRIPTION OF THE DRAWINGS

FIG. 1 is an outer view of an anti-slip garment comprising a lymphedema sleeve and having an anti-slip portion extending upward at the proximal, or upper, end of the garment in an embodiment of the present invention.

FIG. 2 is an inner view of the anti-slip garment shown in FIG. 1, showing a gripping ink applied to the inner surface of the anti-slip portion of the garment.

FIG. 3 is a view of the anti-slip garment shown in FIGS. 1 and 2, showing the garment maintained in a therapeutic position on a wearer's arm.

FIG. 4 is a view of an anti-slip garment comprising a stocking and having an anti-slip portion comprising an upper turned welt extending upward at the proximal end of the garment in an embodiment of the present invention.

### DETAILED DESCRIPTION

As used in this specification, the singular forms “a,” “an,” and “the” include plural referents unless the context clearly dictates otherwise. Thus, for example, the term “an anti-slip portion” is intended to mean a single anti-slip portion or more than one anti-slip portion.

As used in this specification and the appended claims, “proximal” is defined as nearer to a point of reference such as an origin, a point of attachment, or the midline of the body. As used in this specification and the appended claims, “distal” is defined as farther from a point of reference, such as an origin, a point of attachment, or the midline of the body. Thus, as used herein, the words “proximal” and “distal” refer to direction nearer to and farther from, respectively, the upper part of a person's body. That is, the proximal end of an anti-slip garment is nearer the upper part of a wearer's limb, and the distal end of an anti-slip garment is farther from the upper part of a wearer's limb.

Some embodiments of the present invention can include a garment anti-slip mechanism, an anti-slip garment, methods of making such an anti-slip garment, and/or methods of using such an anti-slip garment. Such an anti-slip garment may be, for example, a lymphedema sleeve, a stocking, or other garment design to remain in position about an anatomical structure such as an arm or leg.

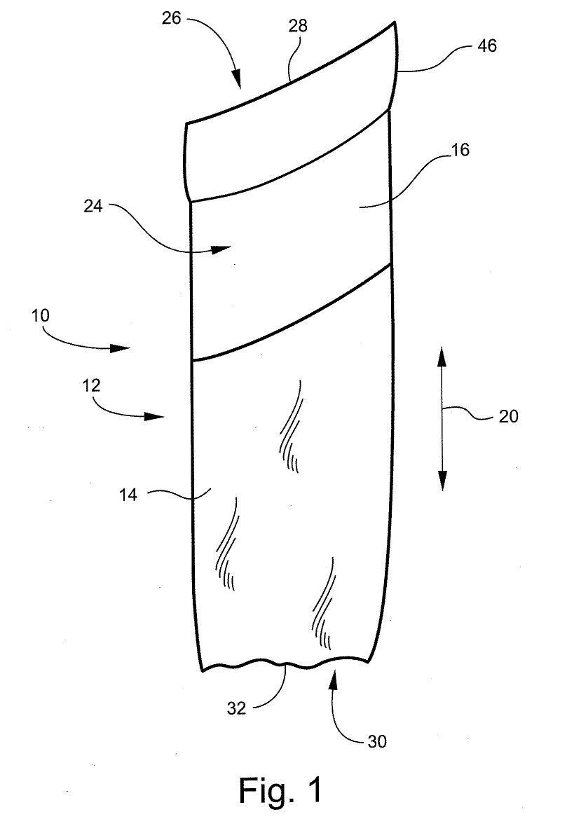

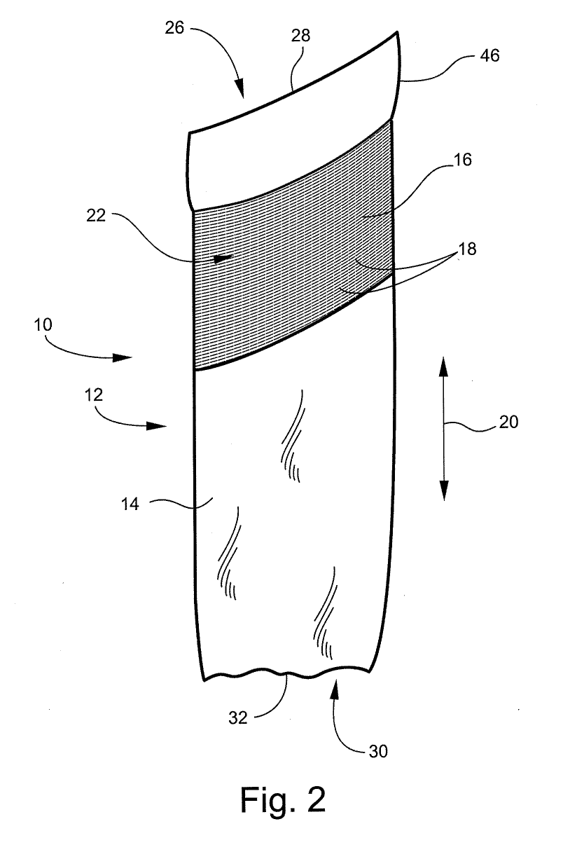

In an illustrative embodiment as shown in FIGS. 1-3, an anti-slip garment 10 comprising a lymphedema sleeve 12 can include a body 14 and an anti-slip portion 16 integrally formed with and extending from the body 14 along the longitudinal axis 20 of the body 14. The lymphedema sleeve body 14 can have dimensions configured to fit the arm of a particular-sized individual. The anti-slip portion 16 can include a gripping composition 18, which can be applied in the form of an ink, that is applied on an inner surface 22 of the fabric of the anti-slip portion 16. In this way, when the sleeve 12 is donned, the gripping ink 18 can contact the wearer's skin. For purposes herein, “gripping ink” and “gripping ink composition” are defined as a composition applicable to a garment as an ink formulation that provides a surface for frictionally engaging a wearer's skin. In this manner, the anti-slip portion 16 can enhance the ability of the garment 10 to be maintained in a desired position on the wearer. FIG. 1 shows the outer surface 24 of the anti-slip garment 10. FIG. 2 shows the inside of the anti-slip garment 10 having the gripping ink 18 applied to the inner surface 22 of the anti-slip portion 16.

In some embodiments, a garment anti-slip mechanism can comprise the application of the gripping ink 18 to the inner surface 22 of the anti-slip portion 16 of the garment 10. In some embodiments, the garment anti-slip mechanism can further include the anti-slip portion 16 being integrally formed with and extending from the body 14 of the garment 10. Such a garment anti-slip mechanism can provide the garment 10 with an enhanced ability to be maintained in a desired position on a wearer.

In an exemplary embodiment, the anti-slip garment 10 can comprise the body 14 and the anti-slip portion 16 integrally formed with the body 14. The anti-slip portion 16 can include the gripping ink composition 18 applied on the inner surface 22 of the anti-slip portion 16. In this way, when the garment 10 is worn, the gripping ink 18 frictionally engages a wearer's skin sufficiently to maintain the garment 10 in a desired position on the wearer.

In some embodiments, the anti-slip portion 16 can be located at or near an upper end 28 of the anti-slip garment 10. In some embodiments, the anti-slip portion 16 can include all or a portion of a welt 46. In some embodiments, the anti-slip garment 10 can comprise a seamless garment. In certain embodiments, the garment body 14 can include a first amount of elasticity and the anti-slip portion 16 can include a second amount of elasticity that is equal to or less than the first amount of elasticity in the body 14. As a result, the risk of constriction due to elasticity in an area underlying the anti-slip portion 16 can be prevented in the event swelling occurs or increases while the garment 10 is being worn.

In some embodiments, the anti-slip garment 10 can further comprise a lymphedema sleeve 12, a stocking 34, a compressive pressure garment, and/or other garment desired to be maintained in a particular position on a wearer.

Conventional medically therapeutic garments, such as lymphedema sleeves and anti-embolism stockings, can include elastomeric materials to provide compressive pressure to the wearer's limb in order to enhance movement of fluid through the limb However, if edema increases in the limb, the degree of compressive pressure from the garment on the limb increases as well. In such conventional therapeutic garments, an upper portion of the garment (such as a sleeve or stocking) can include a greater amount of elasticity than the body of the garment to provide gripping ability to the upper portion in an attempt to keep the upper portion of the garment from riding out of position down the wearer's limb. In such a conventional garment having a more elasticized and thus more highly compressive upper portion, the upper portion can create an even greater compressive pressure on the limb as edema in the limb increases. This can result in constricting the flow of fluid, both intravascular and extravascular lymph fluid, as well as blood, at the point where the upper portion compresses onto the limb.

Use of the gripping composition, such as the gripping ink 18, to provide gripping ability to the anti-slip garment 10 reduces, or eliminates, the need to provide gripping ability with a particular stretch modulus, or elasticity, in the garment 10. This can eliminate the risk of excessive compressive pressure at a point on a wearer, for example, on a wearer's upper arm, in the event the circumference of the arm increases, such as from increasing edema, while the garment is being worn. Thus, in embodiments of the anti-slip garment 10 having a gripping capability provided by the applied gripping ink composition 18 rather than by an elastic component in the gripping portion in an amount in addition to any elastic in the body 14 of the garment 10, the risk of such a constriction point due to the garment design can be prevented.

In some embodiments, the gripping ink 18 can have a friction coefficient sufficient to provide a gripping ability to the ink 18 and to the inside gripping surface 22 of the anti-slip portion 16 onto which it applied. In this manner, the gripping surface 22 of the anti-slip portion 16 can help maintain the garment 10 is a desired position on a wearer. For example, in the illustrative embodiment in FIGS. 1-3 of the lymphedema sleeve 12 having the anti-slip portion 16, the gripping ink 18 applied to the inner surface 22 of the anti-slip portion 16 can have a desired friction coefficient that can help maintain the sleeve 12 on the upper part of a wearer's arm. Such an embodiment can provide the advantage of eliminating the risk that the anti-slip portion 16 would become a constricting point about a wearer's arm in the event of increasing edema in the arm.

In certain embodiments, the gripping ability of the ink 18 can vary depending on the amount of gripping ability desired in a particular garment. The gripping ability of the ink 18 application can be varied by using different ink compositions having various friction coefficients. That is, the gripping ink 18 may comprise a relatively high friction coefficient, a relatively low friction coefficient, or any friction coefficient in the range between a high or low friction coefficient as desired.

In some embodiments, the anti-slip portion 16 can include a plurality of gripping inks 18, at least two of the plurality of gripping inks 18 having a different friction coefficient.

The chemical composition of the ink 18 providing enhanced gripping capability to the anti-slip garment 10 useful in embodiments of the present invention can vary. In certain embodiments, the ink 18 can have gripping properties similar to that of silicone. Silicone may not be successfully applied, for example, as an ink formulation, to the anti-slip garment 10, such as the lymphedema sleeve 12, because the curing temperatures and times necessary for silicone are generally so extreme as to damage the fabric to which silicone is being applied. In embodiments of the present invention, the gripping ink 18 can be applied, for example, by printing, onto a fabric or garment utilizing lower temperatures than, for example, silicone. As a result, the gripping ink 18 applied onto the garment 10 can be cured in less time than silicone or other conventional gripping additives.

In one illustrative embodiment of the present invention, the gripping ink 18 may include a compound comprising silica in a ratio of about 1% to about 5% by weight. One such silica-containing compound known as 10009HDC High Density Clear is available commercially from Polyone Corporation, 8155 Cobb Center Drive, Kennesaw, Ga. Such ink compositions 18 can have advantages over pure elastomeric materials used to provide a gripping surface in a garment. Pure elastomeric materials can be easily cut or nicked and can quickly deteriorate. In addition, pure elastomeric materials can react with skin causing contact dermatitis. Thus, non-silicone ink compositions 18 having gripping capabilities when applied to the garment 10 can provide a more stable, less reactive surface for contacting a wearer and for maintaining the garment 10 in a desired position on the wearer. Other compositions capable of providing garment gripping characteristics on skin, whether an ink or another formulation, may be utilized in some embodiments of the present invention.

In some embodiments, the chemical properties of the gripping ink 18 can vary. That is, in some embodiments, various ink compositions can be applied to the garment 10 in accordance with the present invention for various purposes. For example, the gripping ink 18 can have properties that in a cured state on the garment 10 can be relatively harder or softer and/or can provide more or less gripping capability. In certain embodiments, the gripping ink 18 can have chemical properties that allow and/or facilitate transmission of signals of conditions and/or activities inside the body of a wearer of the garment 10 comprising such ink 18. As an example, some ink compositions 18 useful in particular embodiments can allow and/or facilitate transmission of ultrasound and/or electronic signals through the ink 18 and garment 10 such that physiological conditions and activities, such as blood flow and edema, can be monitored externally through the anti-slip portion 16 of the garment 10. In such embodiments in which the ink 18 allows and/or facilitate transmission of signals through the ink 18 and garment 10, the anti-slip portion 16 can provide sufficient gripping capability to maintain the garment 10 in a desired position so that physiological indicators can be accurately measured through the garment 10.

The ink composition 18 can be applied onto the inside of the garment 10 at various depths. For example, in certain embodiments, the ink 18 can be applied only on the inner surface 22 of the anti-slip portion 16 of the garment 10. In other embodiments, the ink 18 can be applied so as to penetrate half the depth of the garment fabric, the entire depth of the garment fabric, or to other depths as desired.

The gripping ink 18 can be applied onto fabric of the anti-slip garment 10 in various ways. For example, the gripping ink 18 can be applied onto the garment 10 using devices such as an ink jet printer, a gravure printing device, a printing pad, or a screen printing mechanism. Alternatively, the ink 18 may be applied to the garment 10 by spraying or depositing on the garment 10 in other commercially acceptable manners. In some embodiments of the anti-slip garment 10, the gripping ink composition 18 can be permanently fixed to at least the inner surface 22 of the anti-slip portion 16 so as to provide fastness during washing. Accordingly, some embodiments can retain a desired gripping capability over the useful life of the garment 10.

The area of the anti-slip garment 10 covered by the anti-slip portion 16 can vary. For example, in some embodiments, the anti-slip portion 16 can comprise a relatively narrow strip or band about the inside of the circumferential aspect of the garment 10. In such a relatively narrow band, the anti-slip portion 16 can include a relatively small surface area having the gripping ink 18 applied. The gripping ink 18 in such a relatively small surface area can have stronger gripping characteristics than other gripping ink compositions. In other embodiments, the anti-slip portion 16 can include a relatively large surface area having the gripping ink 18 applied. The gripping ink 18 in such a relatively large surface area can have gripping characteristics not as strong as that of other gripping ink compositions. Thus, variation of the surface area covered by the gripping ink 18 and the relative gripping characteristics of the gripping ink 18 can provide a wide range of comfortable, non-constricting anti-slip garments 10. Accordingly, anti-slip garments 10 can be easily customized for various wearers. In an illustrative embodiment, the anti-slip portion 16 can comprise a width along the longitudinal axis 20 of the garment 10 in a range of about one inch to about three inches. In one particular embodiments, the anti-slip portion 16 can comprise a width along the longitudinal axis 20 of the garment 10 of about one and three-fourths inches.

In some embodiments, for example, as shown in FIGS. 1-4, the gripping ink composition 18 can be applied to the proximal end 26, or upper end 28, of the anti-slip garment 10. For example, in the embodiment of the anti-slip lymphedema sleeve 12, the anti-slip portion 16 can be located at and/or near the proximal upper end 28 of the sleeve 12. In an embodiment of the anti-slip stocking 34, the anti-slip portion 16 can be located in the proximal upper thigh area of a thigh-high stocking 34 or in the proximal upper calf area of a knee-high stocking.

In other embodiments, the gripping ink composition 18 can be applied to wearer-contacting surfaces of the garment 10 other than, or in addition to, the proximal 26 or upper end 28. For example, in certain applications the gripping ink 18 can be applied to the proximal upper end 28, to the distal end 30 (lower end 32), to areas of the garment 10 between the proximal upper end 28 and the distal lower end 32, and/or to any combination of desired locations on the garment 10. As an example, in an application in which the anti-slip garment 10 is utilized to cover and/or support a prosthetic arm, the gripping ink 18 can be applied to the upper end 28 of the garment 10 to help maintain the proximal end 26 of the garment 10 in place on a wearer's arm, to the lower end 32 of the garment 10 to help maintain the distal end 30 of the garment 16 in place on the prosthetic limb, and to an area near the middle of the garment 10 to help maintain the garment 10 in place about the juxtaposition of the prosthetic limb to the wearer's natural limb.

The body 14 of the anti-slip garment 10 can comprise natural and/or synthetic yarns known for constructing garments, particularly medically therapeutic garments. For example, the yarn comprising the anti-slip garment body 14 may be cotton or nylon.

Some embodiments of the anti-slip garment 10 comprising the anti-slip portion 16 according to the present invention can include a similar type and amount of elastomeric material(s) as the body 14 of the garment 10. Accordingly, the anti-slip portion 16 of the garment 10 can provide a desired degree of compressive pressure similar to that in the body 14 of the garment 10. In such embodiments, the gripping capability of the anti-slip portion 16 is provided by a gripping composition, such as the gripping ink 18, rather than by an increased elasticity. As a result, in the event of increased edema while the garment 10 is being worn, the anti-slip portion 16 can expand to a degree similar to that of the body 14 of the garment 10 and thus does not cause a constriction of fluid flow underneath the anti-slip portion 16 of the garment 10.

In some embodiments, the amount and/or composition of ink 18 can be adjusted to control the gripping capability, or aggressiveness, of the anti-slip portion 16 of the anti-slip garment 10. That is, in some embodiments of the anti-slip garment 10, the gripping capability can be adjusted by varying the amount of the gripping ink 18 applied to the inner surface 22 of the anti-slip portion 16. In this way, the gripping capability is not limited to the elastic modulus of the anti-slip portion 16. Accordingly, the gripping properties of the anti-slip portion 16 can be easily adjusted to desired degrees appropriate for garments having various circumferences. Stretch properties of the anti-slip portion 16 can be adjusted in the garment construction process, for example, during knitting or weaving. As a result, the gripping anti-slip portion 16 of the garment 10 can comprise a circumference adapted to fit different-sized individuals and/or accommodate an increase in girth of a body portion over which the garment 10 is worn without causing a constriction in that area.

In some embodiments, the anti-slip portion 16 can be integrally formed with the remainder of the anti-slip garment 10, for example, with the body 14 of the garment 10. That is, the lymphedema sleeve 12, compressive pressure stocking, or other garment having the anti-slip portion 16 can be constructed without a seam necessary when a separate elastomeric band is sewn onto, or otherwise attached, to a garment. Accordingly, some embodiments of the anti-slip garment 10 of the present invention can be seamless. This assures that stitching or other seaming materials are not a source for undesirably constricting the garment 10 about a wearer's limb. Some embodiments of the anti-slip garment 10, including the anti-slip portion 16, can be knit. In other embodiments, the anti-slip garment 10, including the anti-slip portion 16, can be woven. In either of such embodiments, the gripping ink composition 18 can be deposited onto the inner surface 22 of the anti-slip portion 16 after the garment 10 is constructed.

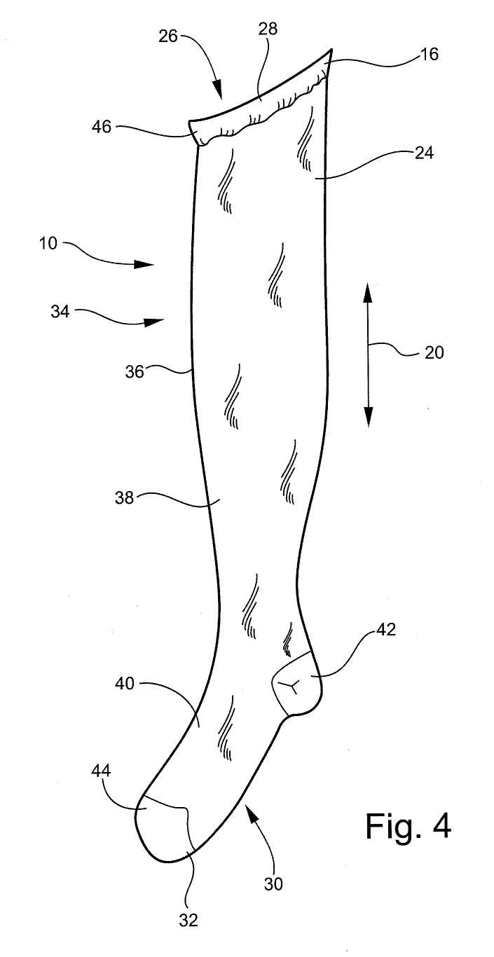

FIG. 4 shows another illustrative embodiment the anti-slip garment 10, which comprises the stocking 34. As shown in FIG. 4, the anti-slip stocking 34 can include a body 36 comprising a leg 38, foot 40, heel 42, and toe 44. The anti-slip portion 16 can be integrally fanned with and extend from the body 36 of the stocking 34 along the longitudinal axis 20 of the body 36. The anti-slip portion 16 can include the gripping composition 18, which can be applied in the form of the gripping ink 18. The gripping ink 18 can be applied on the inner surface (not shown) of the fabric of the anti-slip portion 16 (inside the stocking 34). In this way, when the stocking 34 is donned, the gripping ink 18 can contact the wearer's skin. In this manner, the anti-slip portion 16 can provide the stocking garment 34 with a mechanism for enhancing the ability of the garment 34 to be maintained in a desired position on the leg and foot of the wearer.

While FIG. 4 shows an embodiment of the thigh-high stocking 34, certain embodiments of the anti-slip garment 10 can comprise stockings of different lengths, for example, a knee-high or ankle-high stocking. The stocking body 36 can have dimensions configured to fit the leg and foot of a particular-sized individual. The stocking 36 can be constructed by knitting, weaving, or by other known methods for forming the stocking 36.

In certain embodiments of the present invention, the anti-slip garment 10 can include the welt 46, such as the welt 46 in the lymphedema sleeve 12 shown in FIGS. 1 and 2, and the welt 46 shown in the stocking 34 in FIG. 4. The welt 46 may often be a turned welt 46 as shown in the embodiments in FIGS. 1, 2, and 4. Such a turned welt 46 can be integrally formed, such as by knitting or weaving, with the body 14, 36 of the anti-slip garment 10. In some embodiments, the anti-slip garment 10 can include the welt 46 extending upward at the proximal upper end 28 of the garment 10. In other embodiments, the anti-slip garment 10 can include the welt 46 extending downward at the distal lower end 32 of the garment 10. In still other embodiments, the anti-slip garment 10 can include no welt. In embodiments of the present invention, the anti-slip portion 16 of the garment 10 can comprise all, or a portion, or none of the welt 46.

Some embodiments of the present invention can include methods of making the anti-slip garment 10. For example, such a method can include constructing the body 14 of the anti-slip garment 10, integrally forming the anti-slip portion 16 with the body 14, and applying the gripping ink composition 18 on the inner surface 22 of the anti-slip portion 16. When the garment 10 is worn, the gripping ink 18 frictionally engages a wearer's skin sufficiently to maintain the garment 10 in a desired position on the wearer.

In some embodiments, the anti-slip portion 16 can be integrally formed on the upper end 28 of the anti-slip garment 10. In some embodiments, the integrally formed anti-slip portion 16 can include the welt 46. In some embodiments, the anti-slip garment 10 can be constructed in a seamless manner. In certain embodiments, the garment body 14 can be constructed with a first amount of elasticity, and the anti-slip portion 16 constructed with a second amount of elasticity that is equal to or less than the first amount of elasticity in the body 14. In this way, the risk of constriction due to elasticity in an area underlying the anti-slip portion 16 can be prevented in the event swelling occurs or increases while the garment 10 is being worn.

In some embodiments of such a method of making the anti-slip garment 10, the gripping ink 18 can comprises a silica compound having silica in a ratio of about 1% to about 5% by weight. In some embodiments, the gripping ink 18 can be applied by at least one of ink jet printing, gravure printing, pad printing, screen printing, and spraying. In certain embodiments, a plurality of gripping inks 18 can be applied to the anti-slip portion 16, at least two of the plurality of gripping inks 18 having a different friction coefficient. In certain embodiments, the gripping capability of the anti-slip portion 16 can be adjusted by varying the amount and/or composition of ink 18 applied.

Some embodiments of the present invention can include methods of using the anti-slip garment 10. For example, such a method can include donning the anti-slip garment 10 constructed as described herein, and maintaining the anti-slip garment 10 in a desired position on a wearer. In this way, in the event swelling occurs or increases while the garment 10 is being worn, the risk of constriction in an area underlying the anti-slip portion 16 can be prevented.

Some embodiments of a garment anti-slip mechanism, an anti-slip garment (such as the lymphedema sleeve 12 shown in FIGS. 1-3, and the stocking 34 shown in FIG. 4), methods of making such an anti-slip garment, and/or methods of using a garment anti-slip mechanism and/or anti-slip garment can provide advantages over prior anti-slip garments and/or methods. For example, some embodiments of the present invention can advantageously maintain the garment 10 at a desired position on a wearer without the risk of constricting an area underlying the anti-slip portion 16 if swelling occurs or increases while the garment 10 is being worn.

Another advantage is that in some embodiments the gripping capability of the anti-slip portion 16 can be adjusted. That is, more or less of the gripping ink 18 can be applied to the anti-slip portion 16 of the garment 10 in order to provide an increase or a decreased amount of gripping capability.

Another advantage is that some embodiments the gripping ink composition 18 applied to the anti-slip gripping portion 16 can cause no irritation, or less irritation to a wearer than conventional garment gripping surfaces. For example, the gripping ink 18 can provide desirable gripping capability without applications of silicone, which can possibly cause irritation to a wearer, and which can be expensive.

Another advantage is that in some embodiments stretch properties of the anti-slip portion 16, including, for example, the amount of elastomeric materials included in the anti-slip portion 16, can be varied to accommodate various wearer circumferences, thereby preventing the risk of constriction associated with conventional anti-slip garments that utilize only elastomeric materials to provide gripping capability.

Another advantage is that in some embodiments the anti-slip portion 16 can be integrally formed with the garment 10 so as to provide the anti-slip garment 10 devoid of structures that could cause increased pressure points, such as with band overlaps/joints and/or bulky seams.

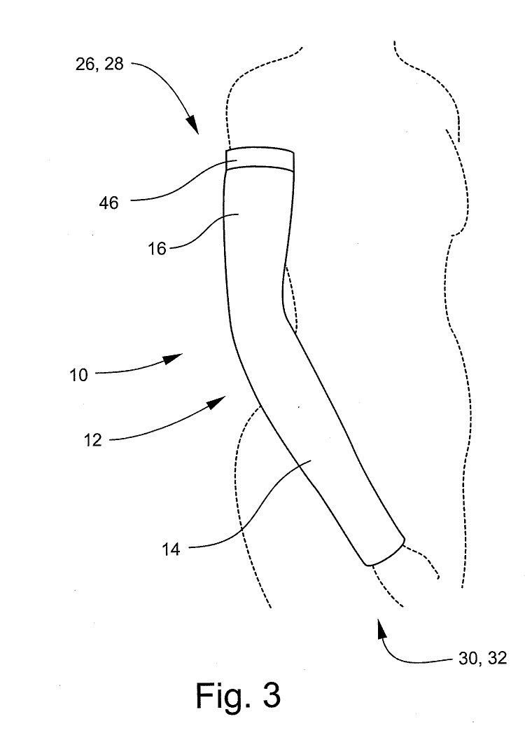

Some embodiments of a garment anti-slip mechanism, an anti-slip garment, methods of making an anti-slip garment, and/or methods of using a garment anti-slip mechanism and/or anti-slip garment can be particularly useful in medical applications. In certain embodiments, an anti-slip composition, such as the gripping ink 18, can be applied to the inner surface 22 of therapeutic medical compression garments. Such compression garments can include garments, such as stockings, sleeves, and the like, for use on a patient to assist in the prevention and/or management of venous or lymphatic disorders and/or thrombosis in the limb of a patient. For example, as shown in FIG. 3, the lymphedema sleeve 12 can include the anti-slip portion 16 comprising the gripping ink 18 applied to the inner surface 22 of the anti-slip portion 16. Such a sleeve 12 can be maintained about the upper portion of a wearer's arm without the risk of constricting the arm if swelling increases while the sleeve is being worn.

Some embodiments of a garment anti-slip mechanism, an anti-slip garment, methods of making an anti-slip garment, and/or methods of using a garment anti-slip mechanism and/or anti-slip garment can be useful in other applications. An anti-slip ink composition 18 as described herein can be applied to any garment in which it is desirable to have the anti-slip portion 16, or gripping portion, that is non-constricting for securing the garment 10 in a position about a wearer. For example, an anti-slip portion 16 having the gripping ink 18 applied on its inner surface 22 can be utilized in sportswear, industrial wear, and/or in fashion wear, including, for example, fashion knee-length and thigh-length stockings, to maintain the garments in position on a wearer.

Although the present invention has been described with reference to particular embodiments, it should be recognized that these embodiments are merely illustrative of the principles of the present invention. Those of ordinary skill in the art will appreciate that a garment anti-slip mechanism, an anti-slip garment, methods of making such an anti-slip garment, and/or methods of using such an anti-slip garment of the present invention may be constructed and implemented in other ways and embodiments. Accordingly, the description herein should not be read as limiting the present invention, as other embodiments also fall within the scope of the present invention.

What is claimed is:

1. An anti-slip garment, comprising:
a body; and
an anti-slip portion integrally formed with the body and comprising a gripping ink composition applied on an inner surface of the anti-slip portion,
wherein when the garment is worn, the gripping ink frictionally engages a wearer's skin sufficiently to maintain the garment in a desired position on the wearer.

2. The anti-slip garment of claim 1, wherein the anti-slip portion further comprises an upper end of the anti-slip garment.

3. The anti-slip garment of claim 1, wherein the anti-slip portion further comprises a welt.

4. The anti-slip garment of claim 1, wherein the anti-slip garment further comprises a seamless garment.

5. The anti-slip garment of claim 1, wherein the body comprises a first amount of elasticity and the anti-slip portion further comprises a second amount of elasticity that is equal to or less than the first amount of elasticity, thereby preventing a risk of constriction due to elasticity in an area underlying the anti-slip portion in the event swelling occurs or increases while the garment is being worn.

6. The anti-slip garment of claim 1, wherein the gripping ink further comprises a compound comprising silica in a ratio of about 1% to about 5% by weight.

7. The anti-slip garment of claim 1, wherein the gripping ink is applied by at least one of an ink jet printer, a gravure printing device, a printing pad, a screen printing mechanism, and a spraying mechanism.

8. The anti-slip garment of claim 1, wherein the anti-slip portion further comprises a plurality of gripping inks, at least two of the plurality of gripping inks having a different friction coefficient.

9. The anti-slip garment of claim 1, wherein the anti-slip garment further comprises a lymphedema sleeve.

10. The anti-slip garment of claim 1, wherein the anti-slip garment further comprises a stocking.

11. The anti-slip garment of claim 1, wherein the anti-slip garment further comprises a compressive pressure garment.

12. A method, comprising:
constructing a body of an anti-slip garment;
integrally forming an anti-slip portion with the body; and
applying a gripping ink composition on an inner surface of the anti-slip portion,
wherein when the garment is worn, the gripping ink frictionally engages a wearer's skin sufficiently to maintain the garment in a desired position on the wearer.

13. The method of claim 12, wherein the integrally forming an anti-slip portion further comprises integrally forming the anti-slip portion on an upper end of the anti-slip garment.

14. The method of claim 12, wherein the integrally forming an anti-slip portion further comprises integrally forming a welt.

15. The method of claim 12, further comprising constructing the anti-slip garment in a seamless manner.

16. The method of claim 12, further comprising constructing the body with a first amount of elasticity and integrally forming the anti-slip portion with a second amount of elasticity that is equal to or less than the first amount of elasticity, thereby preventing a risk of constriction due to elasticity in an area underlying the anti-slip portion in the event swelling occurs or increases while the garment is being worn.

17. The method of claim 12, wherein the gripping ink comprises a compound comprising silica in a ratio of about 1% to about 5% by weight.

18. The method of claim 12, wherein the applying a gripping ink composition further comprises applying the gripping ink by at least one of ink jet printing, gravure printing, pad printing, screen printing, and spraying.

19. The method of claim 12, wherein the applying a gripping ink composition further comprises applying a plurality of gripping inks, at least two of the plurality of gripping inks having a different friction coefficient.

20. The method of claim 12, further comprising adjusting a gripping capability of the anti-slip portion by varying an amount and/or composition of ink applied.
